(19) United States
(12) Patent Application Publication
(10) Pub. No.: US 2009/0069904 A1
(43) Pub. Date: Mar. 12, 2009

(54) BIOMATERIAL INCLUDING MICROPORES

(75) Inventor: George J. Picha, Independence, OH (US)

Correspondence Address: PEARNE & GORDON LLP, 1801 EAST 9TH STREET, SUITE 1200, CLEVELAND, OH, 44114-3108, US

(73) Assignee: Applied Medical Research, Brecksville, OH (US)

(21) Appl. No.: 12/209,370

(22) Filed: Sep. 12, 2008

Related U.S. Application Data
(60) Provisional application No. 60/971,706, filed on Sep. 12, 2007.

(51) Int. Cl. A61F 2/02

(52) U.S. Cl. 623/23.72; 623/23.74; 623/23.75

(57) ABSTRACT

A biomaterial including a designed pattern of micropores one at least one surface of the biomaterial is described. The micropores can be provided in a regular or irregular pattern, and can be either continuous or discontinuous. The biomaterial may be formed from a variety of materials, such as a biocompatible polymer or biocompatible tissue. The biomaterial including micropores on a surface may be used for a variety of medical applications such as tissue scaffolding, drug delivery, or tissue fixation.

### CONTINUING APPLICATION DATA

This application claims the benefit of U.S. Provisional Application Ser. No. 60/971,706, filed Sep. 12, 2007, which is incorporated by reference herein.

### BACKGROUND

When a biomaterial is placed in a physiological environment, it is exposed to a variety of biochemical and immunological processes intended to remove or neutralize the foreign material. These processes can occur even when biomaterial that is physiologically inert is introduced. These processes include protein fouling, degradation and dissolution, and calcification. For example, moments after implantation, biomaterials are surrounded by adsorbed proteins that fill the wound site or are secreted by cells associated with wound healing. Adsorption of local proteins is rapidly followed by the subsequent arrival of humoral factors such as antibodies and leukocytes, which further alter the surface of the biomaterial. In turn, protein adsorption can result in changes in microenvironmental conditions that facilitate degradation of the biomaterial and/or deposition of calcium-containing materials. Over time, this cascade of reactions can lead to a variety of undesirable physiological effects.

A variety of techniques have been used to attempt to counteract the undesired physiological response to implanted biomaterials. These include physiochemical modification, biological modification, and morphological modification. In physiochemical modification, surface energy, surface charge, or surface composition were altered. For example, glow discharge, in which surfaces are exposed to an ionizing inert gas, may be used to increase the surface free energy of many metals and polymers. The greater reactivity of surfaces with higher surface energy leads to increased tissue adhesion, leading to a more natural physiological integration that diminishes fibrous encapsulation. Biological modification, on the other hand, includes techniques such as the deposition of RGD-containing peptides on the on the biomaterial surface to enhance integration of the biomaterial with the extracellular protein matrix. Other examples of biological modification include the immobilization of growth factors such as epidermal growth factor, insulin-like growth factor I, and/or bone morphogenic protein 4 on biomaterial surfaces to induce specific cellular responses.

Alteration of surface morphology and roughness has also been used to influence the physiological response to the implantation of biomaterial. For example, it has been demonstrated that substantial improvement of the vascularization near an implanted biomaterial and a concurrent reduction in fibrous capsule formation can be achieved by providing an array of small, closely spaced projections on a biomaterial surface. These surface projections interfere with the formation of long, uniform bundles of collagen fibers, a process referred to as long-range collagen ordering that is involved in fibrous capsule formation.

Various textured surfaces have been proposed for use on implantable devices to help decrease fibrous capsule formation. For example, U.S. Pat. No. 4,960,425 to Yan et al. discloses a surgical prosthesis with a textured surface consisting of a plurality substantially microscopic peaks and valleys, which are preferably placed on a reticulated foam. U.S. Pat. No. 4,955,909 to Ersek et al., on the other hand, teaches a textured silicone implant including randomly places pillars of a non-uniform height and width. Furthermore, U.S. Pat. No. 5,207,709 to Picha provides an implantable medical device including a plurality of fin projections arrayed in a basket weave or herringbone weave-like pattern.

Polymer surfaces may be textured as a result of foaming the polymer. A number of patents describe the preparation of polymeric foams. For example, U.S. Pat. No. 5,969,020 describes the preparation of microcellular foams having a continuous, uniform open-cell structure prepared by the removal of crystalline “fugitive material,” leading to the formation of voids or pores throughout the material with a diameter of about 1 to 200 microns. These types of foams can be prepared using either biodegradable or non-biodegradable polymers, and are typically used as scaffolding material or for drug delivery. An additional variant on this type of foamed polymer is supplied by patent application Ser. No. 10/106,007, by Brown et al., which describes a biocompatible polymeric foam scaffold that includes a network of branched channels that are effective to encourage vascularization and tissue growth within the scaffold in which the branched channels are formed as a result of branched crystal formation. These polymeric foams are sponge-like materials with channels that run throughout material. Foaming alters the composition of the biomaterial used, and is not available for all types of suitable biomaterials. However, the pores formed by this method tend to vary in size and length, and thus do not have an easily predictable or necessarily positive effect on biomaterial biocompatibility.

### SUMMARY OF THE INVENTION

In one aspect, the present invention provides a biomaterial including a substrate that includes a designed pattern of micropores on one or more surfaces of the substrate. In one embodiment, the substrate includes a biocompatible polymer. In embodiments including a biocompatible polymer, the biocompatible polymer may further include a biodegradable polymer. In further embodiments, the biodegradable polymer can be selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups poly(anhydrides), polyphosphazenes, tyrosine derived polycarbonates, and biopolymers.

In an additional embodiment, the micropores include continuous micropores. In further embodiments, the diameter of the micropores is from about 10 microns to about 5,000 microns. In another embodiment, the diameter of the micropores is from about 100 to about 1,000 microns. In further embodiments, the surface of the substrate further includes texturing.

In another embodiment of the biomaterial, the micropores retain a pharmacologically active agent. In further embodiments of the invention that include a pharmacologically active agent, the agent can be retained in the micropores by a retaining material. Alternately, or in addition, the biomaterial including a pharmacologically active agent can be provided with a laminate layer one or more surface of the substrate over the micropores.

In a further embodiment of the biomaterial, the designed pattern includes an uneven pattern of spaced micropores that allow for asymmetrical expansion of the biomaterial. In another embodiment, the designed pattern of micropores is a regular pattern. In yet another embodiment, the designed pattern of micropores is a gradient pattern.

The biomaterial can be provided in a variety of shapes. For example, in one embodiment the biomaterial is provided as a sheet. The biomaterial can also include a plurality of layers of biocompatible polymers. In another embodiment, the biomaterial is provided as a tissue scaffold.

Another aspect of the present invention provides a method of tissue reconstruction that includes surgically exposing a location in an individual's body where tissue is to be reconstructed and placing a tissue scaffold including the biomaterial of the invention at that location. In one embodiment, the tissue to be reconstructed is cartilage. In further embodiments, the tissue includes a nose or an ear.

The terms “comprises” and variations thereof do not have a limiting meaning where these terms appear in the description and claims. Unless otherwise specified, “a,” “an,” “the,” and “at least one” are used interchangeably and mean one or more than one. Thus, for example, a composition that comprises “a” type of cell can be interpreted to mean that the composition includes “one or more” types of cells. Similarly, a composition comprising “a” polymer can be interpreted to mean that the composition includes “one or more” polymers. Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 provides a perspective view of a bent sheet of biomaterial that includes a designed pattern of regularly spaced continuous micropores.

FIG. 2 provides a perspective view of a flat sheet of biomaterial that includes a mixture of two different types of micropores.

FIG. 3 provides a perspective view of a flat sheet of biomaterial that includes a designed pattern of cylindrical micropores as well as a plurality of pillars contiguous with the porous surface of the biomaterial.

FIG. 4 provides a cross-sectional view of a sheet of biomaterial that includes a micropore with an expanded mid-section including a layer of a pharmacologically active agent.

FIG. 5 provides a cross-sectional view of a sheet of biomaterial that includes micropores and laminated sheets to retain a pharmacologically active agent.

### DETAILED DESCRIPTION OF ILLUSTRATIVE EMBODIMENTS

The present invention provides a biocompatible biomaterial that includes a designed pattern of micropores on one or more surfaces of the substrate forming the biomaterial. The designed pattern of micropores preferably improves the biocompatibility of the biomaterial by, for example, encouraging biomaterial integration, encouraging capillary ingrowth, and/or providing pharmacological agents or suspended cells. In the following description, for purposes of explanation, numerous specific details are set forth in order to provide a thorough understanding of the present invention. It may be evident, however, that the present invention may be practiced without these specific details.

The micropores provided on the surface of the substrate are micropores that have a diameter from about 10 microns or more. For example, the micropores may have a diameter from about 10 microns to about 5,000 microns, from about 100 microns to about 1,000 microns, or from about 200 microns to about 600 microns. In various embodiments, the micropores can occupy a different amount of the surface area of the substrate. For example, the micropores can occupy 10% to 25% of the surface, 25% to 50% of the surface, 50% to 75% of the surface, or greater than 75% of the surface area of the substrate forming the biomaterial. Increasing the surface area occupied by the micropores decreases the amount of the surface area of the biomaterial that directly interacts with adjacent tissue, which can improve the biocompatibility of the biomaterial. In addition, even after tissue ingrowth has occurred, biomaterial including micropores will have less volume as compared to biomaterial lacking micropores, and can thus present less foreign material.

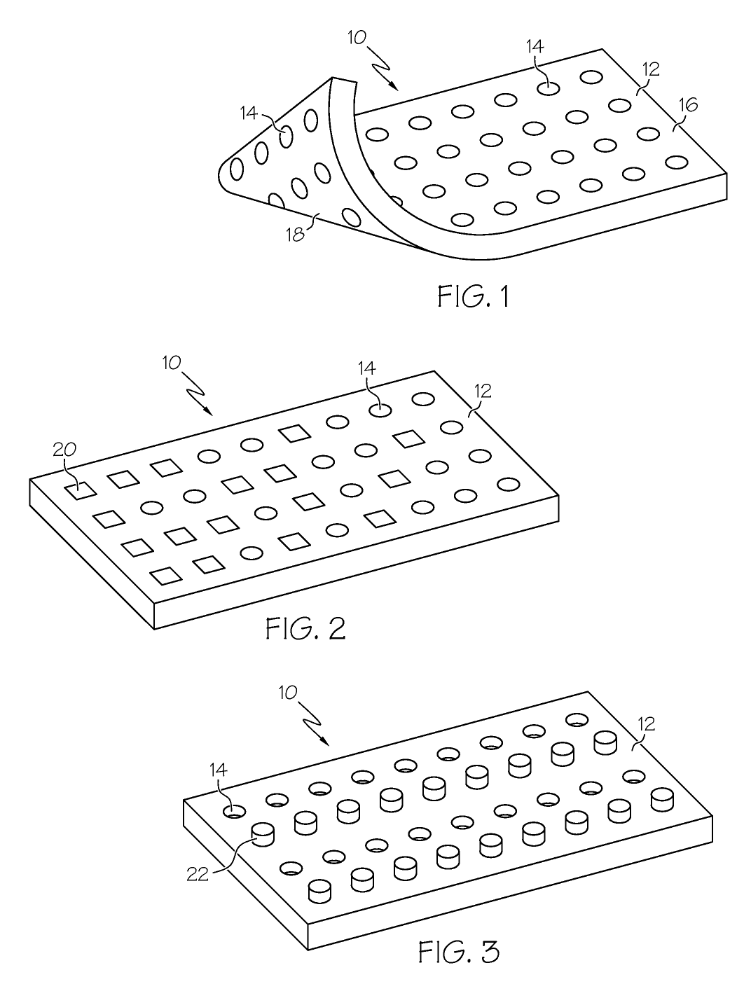

The micropores provided in the biomaterial can be continuous micropores. A biomaterial including a regular pattern of continuous micropores is shown in FIG. 1. The biomaterial 10 is formed of a substrate 12 that includes a regular pattern of micropores 14 on one or more sides of the biomaterial 10. A continuous micropore 14 forms a channel through the biomaterial, with an opening at each end (i.e., two openings). For example, a sheet of biomaterial 10 can include micropores 14 that open on one side of the substrate (e.g., the top surface 16 of the substrate 12) and then pass through the biomaterial to also open on another side (e.g., the bottom surface 18 of the substrate 12). The biomaterial 10 shown in FIG. 1 is folded at one end so that the top surface 16 and the bottom surface 18 of the substrate 12 can both be seen, to help demonstrate the continuous nature of the micropores 14 in this embodiment. While a sheet of biomaterial provides an example of the use of continuous micropores that can be readily envisioned, it should be understood that the biomaterial can be provided in other forms, and that these other forms can also include continuous micropores that pass from one side of the biomaterial to another. Preferably, the continuous micropores are relatively linear and pass directly from one side of the biomaterial to the other.

Alternately, or in addition, the biomaterial can include micropores that are discontinuous. Discontinuous micropores have an opening on one side of the biomaterial but simply provide a concave region on the surface of the biomaterial and do not provide a channel to another side of the biomaterial. Discontinuous micropores can serve to limit tissue vascularity. If biodegradable material is used in the biomaterial, or discontinuous micropores are provided that include a biodegradable membrane, discontinuous micropores can become continuous after a sufficient period in vivo.

The micropores provided on one or more surfaces of the substrate of the biomaterial are provided in a designed pattern. A designed pattern includes a plurality of micropores that are provided on a surface in accordance with a predetermined plan. Accordingly, the micropores are preferably provided on the surface of the substrate in a non-random fashion. The designed pattern can be regular or irregular. If a regular pattern is used, the micropores may be provided with an even distance between the micropores. For example, the distance from one micropore to the next micropore may be from about 25 microns to about 1,000 microns, or from about 25 microns to 100 microns.

If an irregular pattern is used, the spacing from one micropore to the next will differ from one region of the biomaterial to another. For example, in one embodiment with an irregular pattern, the micropores can be spaced apart from one another in a gradient fashion such that the distance between micropores increases or decreases along the surface of the biomaterial. A biomaterial that includes a gradient pattern is shown in FIG. 2. The biomaterial 10 shown in FIG. 2 is formed of a substrate 12 that includes a micropore 14 of a first type (e.g., a circular micropore), and also includes a variant micropore 20 of a second type (e.g., a square micropore). The number of variant micropores changes as you move from one end of the biomaterial to the other, resulting in a gradient pattern.

In embodiments in which the micropores are provided in an irregular pattern (e.g., micropores that are spaced apart in a gradient fashion), this designed pattern may alter the properties of the biomaterial in a varying fashion that allows for the asymmetrical expansion of the biomaterial when the biomaterial is stretched or otherwise subject to mechanical stress. The irregular pattern may be created by providing micropores in one region of the surface of the biomaterial may have a different diameter or shape from the micropores found on a different region of the surface of the biomaterial. Alternately, it may be provided by changing the distance between micropores in different regions of the biomaterial. In further embodiments, a variety of sizes of micropores may be intermixed on the surface of the biomaterial. While the invention includes biomaterials with micropores that have a regular, circular diameter, the micropores may also have an irregular diameter, in which case the pore diameter represents the average cross-sectional distance of the pore.

The micropores can be provided in any suitable shape. The micropores are typically a column that runs through the substrate with an opening on each side. The walls of the column can be fairly straight, or they can bow inwards, outwards to form a somewhat spherical column, or angled to form a cone-shaped column. If the micropores are discontinuous, they may also form a concave opening that does not pass through the substrate. The circumference of the micropores can also vary in shape. For example, the micropores can have a round circumference, or the circumference can follow various polygonal shapes such as square, rectangular, hexagonal, etc. An example of a simple micropore shape is a straight round channel; i.e., a round, cylindrical-shaped column. A biomaterial can also include combinations of different shapes of micropores. For example, the substrate of the biomaterial could include a combination of square and circular micropores.

The micropores provided on the surface of the substrate can improve the biocompatibility of the biomaterial. While not intending to be bound by theory, it is believed that the micropores improve the biocompatibility of the biomaterial though one or more of the following mechanisms. For example, it is believed that micropores of a sufficient diameter (e.g., of 100 microns or more) can encourage vascular ingrowth into the biomaterial. Micropores can also encourage capsule formation to occur within the pores, which disrupts excessive capsule formation and decreases the chance of infection. For example, infection may be discouraged as a result of increased tissue ingrowth with provides an integrated morphology that is more resistant to infection. In addition, micropores can help reduce the chance of contracture around an implant formed of biomaterial including micropores. The micropores may also improve biocompatibility as a result of minimizing the amount of foreign material present on the surface of an implant, as a result of the decreased surface area resulting from the inclusion of micropores on the surface.

Another method by which the biomaterial can improve biocompatibility is through encouraging fibrous ingrowth. Fibrous ingrowth also facilitates implant fixation as a result of the anchoring effect of fibrous growth into the biomaterial. The micropores encourage fibrous ingrowth partly as a result of allowing cell migration into the micropores. Cell migration and fibrous ingrowth are best encouraged by continuous micropores. Discontinuous micropores, on the other hand, while having a number of positive effects on biomaterial biocompatibility, tend to inhibit fibroplasia and cell migration into the micropores, particularly discontinuous micropores.

The biomaterial can also be provided with texturing on one or more surfaces of the substrate. Texturing includes various features provided on the surface of the substrate that extend outwards from the surface rather than forming openings in the surface. For example, surface texturing may include a plurality of texturing objects 22 such as pillars, continuous or discontinuous, projecting from on one or more surfaces of the substrate. An example of a biomaterial of the present invention that includes both a designed pattern of micropores and texturing is provided by FIG. 3, which provides a perspective view of a flat sheet of biomaterial 10 that includes a designed pattern of cylindrical micropores 14 as well as a plurality of texturing objects 22 (e.g., pillars) extending from the surface of the substrate 12 of the biomaterial 10.

The texturing objects can be randomly dispersed along the surface, or they may be placed in a regular or irregular designed pattern. The texturing objects can be employed for increase fixation of the biomaterial on or within the implant site. The texturing objects can be positioned between the micropores provided through the substrate and can be of any suitable shape and size depending upon the particular application intended for the biomaterial. For example, the texturing objects may have a circular, square, rectangular, or triangular cross-section, although a roughly circular cross-section is preferred. The outwardly projecting texturing objects can also significantly disrupt subsequent fibroplasia on the surface of the substrate of the biomaterial.

The height of the texturing objects ranges from about 50 microns to about 5000 microns. It is preferred that the height of the texturing objects ranges from about 400 microns to about 3,300 microns, and more preferably from about 750 microns to about 1600 microns. More preferably, the height of the texturing objects is selected in combination with the width of the texturing objects. The width of the texturing objects ranges from about 50 microns to about 1000 microns. It is preferred that the width of the texturing objects ranges from about 100 microns to about 900 microns, and more preferably from about 250 microns to about 800 microns. Further details regarding the nature of the texturing objects that may be included on the surface of the biomaterial, such as a thickened base portion and rounded tops, may be found in U.S. Pat. No. 6,106,558, which is incorporated herein by reference.

The micropores may be provided on one or more surfaces of the biomaterial. For example, a flat sheet of biomaterial may include continuous micropores with openings on both sides of the sheet. Alternately, when discontinuous micropores are used, they can be provided on a single surface or they can be provided on more than one surface of the biomaterial. The micropores provided can be the same on both surfaces, or the type of micropores and/or the designed pattern governing the placement of the micropores may differ from one surface to another on the biomaterial. Use of differing micropores may provide the biomaterial with useful characteristics, such as the ability to deliver drugs from one side of the biomaterial while encouraging tissue attachment along another side of the biomaterial. Continuous micropores can also be modified to provide different characteristics at different surfaces by, for example, altering the size or shape of the opening on one side compared with the other.

The biomaterial can be provided in a wide variety of dimensions and can be of any suitable size and shape and can be configured in any suitable manner. For example, the biomaterial may be provided as a cylinder, a flat sheet, a twisted structure such as a helix, branched tubular shapes, spherical shapes, hemispherical shapes, or conical shapes. The shape of the biomaterial will vary depending upon its intended use and the manufacturing process used. For example, when the biomaterial is designed for use as a tissue scaffold material, the biomaterial may be provided as an organ-shaped or tissue-shaped material where the shape used facilitates the regeneration or repair of the desired organ or tissue. For example, a ridge or cone shape may be suitable for repair of a nose or ear, while a spherical or hemispherical shape may be useful if the biomaterial is designed for reconstruction or repair of an internal organ such as a bladder or pericardium.

The biomaterial used can be of any suitable stiffness (e.g., durometer) and toughness that ranges from soft and flexible to hard and rigid, depending upon the needs of the surgeon, the tissue environment, and the support requirements. The biomaterial can be configured such that it can be stretched in one direction more easily than in another direction. For instance, the substrate can be composed of one or more of an elastomer, plastic, ceramic, metal, biopolymers, polymers, tissue engineered tissue, or naturally occurring tissue such as bone, cartilage, fascia. Preferably, the substrate is a biocompatible polymer, biocompatible animal tissue, or a biodegradable polymer.

A wide variety of biocompatible polymers can be included in the biomaterial. As used herein, the term polymer also includes homopolymers, copolymers, terpolymers, interpolymers and blends thereof. A biocompatible polymer, as used herein, is a polymer that produces little if any adverse biological response when used in vivo.

Examples of suitable biocompatible polymers include non-absorbable polymers such as polypropylene, polyethylene, polyethylene terephthalate, polytetrafluoroethylene, polyaryletherketone, nylon, fluorinated ethylene propylene, polybutester, and silicone, or copolymers thereof (e.g., a copolymer of polypropylene and polyethylene); absorbable polymers such as polyglycolic acid (PGA), polylactic acid (PLA), polycaprolactone, and polyhydroxyalkanoate, copolymers thereof (e.g., a copolymer of PGA and PLA), and mixtures thereof.

A wide variety of biodegradable polymers can also be used. Biodegradable polymers, as defined herein, are a subset of biocompatible polymers that gradually disintegrate or are absorbed in vivo over a period of time (e.g., within months or years). Examples of suitable biodegradable polymers include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups poly(anhydrides), polyphosphazenes, tyrosine derived polycarbonates, collagen, poly(alpha esters) such as poly(lactate acid) and polyglycolic acid, polyanhydrides, and various mixtures thereof,

Preferred biodegradable polymer material includes polyglycolic acid and polyglactin. Other biodegradable materials include cellulose ether, cellulose, cellulosic ester, fluorinated polyethylene, phenolic, poly-4-methylpentene, polyacrylonitrile, polyamide, polyamideimide, polyacrylate, polybenzoxazole, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polycaprolactone, polysulfide, polysulfone, polytetrafluoroethylene, polythioether, polytriazole, polyurethane, polyvinyl (e.g., polyvinyl alcohol), polyvinylidene fluoride, regenerated cellulose, silicone, urea-formaldehyde, or copolymers or physical blends of these materials.

The substrate used to form the biomaterial can also include micropores within the substrate, which may be referred to herein as “interior micropores.” As described herein, interior micropores are submerged beneath the surface of the material and represent voids with no openings on the surface. Alternately, or in addition, the biomaterial may be formed from a foamed biocompatible and/or a biodegradable polymer that include a highly interconnected set of interior micropores and/or channels, which may or may not connect with the surface of the biomaterial. Foamed biomaterial provides several additional advantages, such as decreasing fibroplasia, increasing angiogenesis, and/or changing the predominant inflammatory cell type present (e.g., inducing more foreign body giant cells). These advantages can complement those provided by the micropores provided in the biomaterial.

The interior micropores can vary in size. In some embodiments, they may have a diameter from about 10 microns to about 1,000 microns. Alternately, the interior micro pores may have a diameter from about 50 microns to about 750 microns, from about 100 microns to about 600 microns, or from about 200 microns to about 500 microns. In addition to varying in size, they may also vary in quantity, as noted above, to provide differing levels of porosity. For example, the micropores may provide a porosity of about 25%, about 50%, or about 75% or more.

The biomaterial can also include biocompatible animal tissue. Preferably the biocompatible animal tissue is decellularized animal tissue. Decellularized animal tissue is animal tissue that has been decellularized under conditions and for sufficient times so that antigenic cells and cellular components are substantially removed, leaving a biomaterial consisting primarily of extracellular matrix components such as collagen and elastin. Decellularization may be accomplished using any of a variety of detergents, emulsification agents, proteases, and/or high or low ionic strength solutions using procedures known to those skilled in the art, such as the procedures described in U.S. Pat. No. 6,962,814, the disclosure of which is incorporated herein by reference. Animal tissue may be obtained from a wide variety of animals, including humans, and can be selected from various different tissues. Preferred animal tissues include muscle and bone.

The substrate of the biomaterial can also be a composite of biocompatible animal tissue and a polymer. Examples of such composite materials are described in U.S. patent application Ser. No. 10/509,216, the disclosure of which is incorporated herein by reference. The substrate of the biomaterial can also use polymer blends that transition from one composition to another in a gradient fashion to provide anisotropic properties. The biomaterial can also include one or more layers of differing biocompatible substrate materials (e.g., biodegradable polymers) and/or biocompatible animal tissue. For example, the biomaterial can be formed as a multi-layered system in which one layer is provided for structure (e.g., a base layer) and another layer is provided for controlling a bio interaction (e.g., a bioactive layer). Any number of suitable layers for any suitable purposed can be utilized and is contemplated as falling within the scope of the present invention.

The biomaterial substrate can also be a biocompatible ceramic or metal. Ceramic or metal biomaterial are well suited for applications where various types of structural strength (e.g., compressive strength or tensile strength). Examples of biocompatible ceramics include alumina, zirconia, pyrolytic carbon, bioglass, hydroxyapatite, and calcium phosphate (e.g., tricalcium phosphate). Examples of biocompatible metals include stainless steel, titanium (e.g., Ti 6Al-4V), nickel, cobalt, chrome, niobium, molybdenum, zirconium, tantalum, and combinations thereof. Micropores can be formed in a biocompatible ceramic or metal using many of the same methods used to provide micropores in a polymeric biomaterial, such described herein.

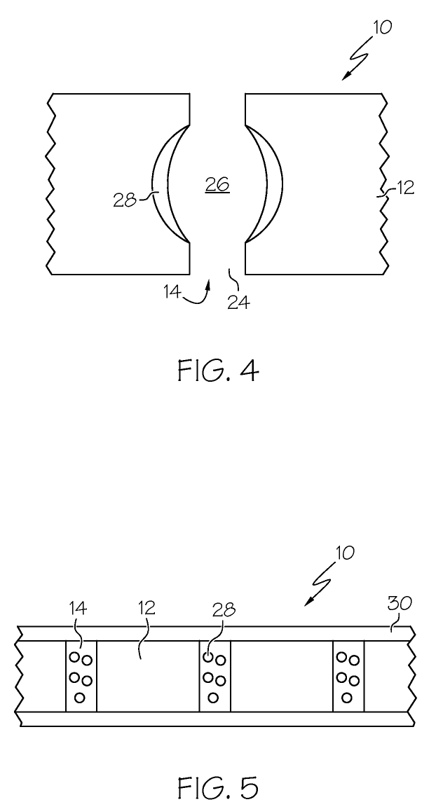

The micropores of the biomaterial may have a configuration that is effective to retain and deliver a pharmacologically active agent. For example, micropores suitable for retaining pharmacologically active agents include both continuous and discontinuous micropores. An example of a continuous micropore that is configured to increase retention of a pharmacologically active agent is shown in FIG. 4, which provides a cross-sectional view of a sheet of biomaterial 10 that includes a micropore 14 with regular cylindrical openings 24, an expanded spherical mid-section 26, and a layer of a pharmacologically active agent 28 retained within the spherical mid-section 26. However, while such a shape may help retain a pharmacologically active agent, it is not necessary to use a micropore having this shape to retain a pharmacologically active agent and other micropore shapes are suitable as well.

Any suitable pattern or array of filling the pores can be employed. For example, pharmacologically active agents and/or cells may be introduced to the biomaterial by, for example, soaking the biomaterial in a solution containing the pharmacologically active agent and/or cells. The micropores can also be selectively filled with one or more different pharmacologically active agent for selective drug delivery. For instance, one pharmacologically active agent can be provided within pores located about a periphery of the structure and another pharmacologically active agent can be provided within pores located in a central portion of the structure.

Various means can be used to help retain the pharmacologically active agent within the micropores. For example, the pharmacologically active agent can be allowed to dry within the micropores. In another embodiment of the invention, the pharmacologically active agent can be suspended in a retaining material, such as a biodegradable polymer, which is then used to fill the micropores. By using a low-melt polymer (e.g., polyethylene glycol) or by later cross-linking the polymer, the material can be delivered into the micropores and then fixed in place where it will help retain the pharmacologically active agent. These methods can be readily used to include a drug of choice into the biomaterial at the time of surgery, shortly before the biomaterial is implanted. The release rate can be varied by using retaining materials that release the pharmacologically active agent at the desired rate.

Another method for retaining a pharmacologically active agent is the use of a laminate layer over the biomaterial to help retain the pharmacologically active agent. A biomaterial including a laminate layer is illustrated in FIG. 5. As seen in the figure, the biomaterial 10 includes a substrate 12 (e.g., a biocompatible polymer) that includes a number of micropores 14. The micropores 14 are filled (all or in part) with a pharmacologically active agent 28. The pharmacologically active agent 28 is then retained within the micropores 14 by one or more laminate layers 30 that are placed on one or more surfaces of the substrate 12. The laminate layer 30 can be a material that retains the pharmacologically active agent 28, while also allowing its eventual release in vivo. For example, the laminate layer 30 can be a biodegradable polymer that releases the pharmacologically active agent from the micropores 14 as it degrades. Alternately, or in addition, the laminate layer 30 can be a biocompatible material that allows the pharmacologically active agent 28 to gradually elute from the micropores 14 by passing through the laminate layer 30. The release rate can be varied depending on the choice of material for the laminating layer and the thickness of the laminating layer.

Pharmacologically active agents, as used herein, broadly includes physiologically or pharmacologically active substances that are intended to produce a localized effect at the administration site or a systemic effect at a site remote from the administration site. Such agents include inorganic and organic compounds, for example, pharmacological agents that act on the central nervous system such as hypnotics and sedatives, psychic energizers, tranquilizers, anticonvulsants, muscle relaxants and anti-Parkinson agents, antipyretics and anti-inflammatory agents, local anaesthetics, anti-spasmodics and anti-ulcer agents, prostaglandins, antibiotics, hormonal agents, steroids that affect fibroblasts/fibroblast sites, steroids that affect osteoblasts/osteoblast sites in fibroplasia, estrogenic steroids, progestational steroids, such as for contraceptive purposes, sympathomimetic drugs, cardiovascular drugs, diuretics, antiparasitic agents, antiviral agents, antifungal agents, hypoglycemic drugs and ophthalmic drugs.

Depending on the particular application, certain types of pharmacologically active agents may be preferred. For example, biomaterial intended for use as a tissue scaffold material may include pharmacologically active agents that that substantially modify (by suppressing or promoting) tissue adhesion and/or tissue growth. Suitable growth factors include cytokines, interleukins, and other peptide growth factors such as epidermal growth factor (EGF), members of the fibroblast growth factor (FGF) family, platelet-derived growth factor (PDGF), nerve growth factor (NGF), glial growth factor (GGF), vascular endothelial growth factor (VEGF), or members of the Transforming Growth Factor (TGF) family (e.g., TGF-α or TGF-β). Various other additives such as diluents, carriers, stabilizers, and other excipients may be included with the pharmacologically active agent.

Alternatively, or in addition, the micropores of the biomaterial can be configured to retain one or more types of biological cells. For example, the micropores can contain stem cells, progenitor cells (e.g., osteoblasts or any other partially differentiated cell), cells of an established cell line, or mature cells such as myocytes, adipocytes, fibroblasts, hepatocytes, and chondrocytes. The cells can be harvested from a patient and then processed to provide a specific cell type. The cells can then be seeded onto the structure in vitro, and the biomaterial later implanted once the cells have grown into the microporous structure of the biomaterial.

The micropores and/or one or more surfaces of the substrate of the biomaterial (e.g., a surface that comes into contact with cells, tissues, or organs upon implantation in a subject) may contain or be coated with one or more molecules involved in cell-cell adhesion or cell-matrix adhesion (i.e., an adhesion ligand). The adhesion ligand can be an adheren or cadherin and, more specifically, can be of the ICAM (intercellular adhesion molecule) family or the N-CAM (neural cell adhesion molecule) family of proteins. The adhesion ligands can be used to facilitate integrin-dependent migration of cells, such as fibroblasts and endothelial cells, to and into the biomaterial. Growth factors located therein could also induce the desired differentiation and/or proliferation and differentiation of the cells (e.g., stem cells or progenitor cells) included in the biomaterial. The biomaterial including micropores on least one surface of the substrate can thus encourage cellular invasion and provide morphogenic signals to attract and retain endogenous or exogenous progenitor cells and induce their differentiation to a tissue specific pathway, as well as encouraging development of vasculature.

The porosity of the biomaterial can be configured to allow for asymmetric expansion and/or anisotropic behaviour of the biomaterial. This may be achieved by unevenly distributing the micropores on or within the biomaterial or providing continuous micropores that vary in diameter to provide the biomaterial with varying properties such as asymmetric expansion or flexibility. For example, an increasing number of micropores can be provided along a gradient on or within the biomaterial. Such an increasing gradient of micropores could provide a biomaterial that is lighter in weight or more flexible at one end relative to the other, or provide varying quantities of pharmacologically active agent in different regions of the biomaterial. Alternately, or in addition, regions of micropores of different sizes can also be used to provide the biomaterial with anisotropic properties. The different numbers or different sizes of micropores can be provided in a number of different ways. For example, layers of biomaterial may be prepared that include varying amounts or sizes of micropores. As another example, the mold used to produce the biomaterial may be crafted such that it contains regions of micropores that vary in size or number.

The micropores can be formed by any acceptable process. For example, the micropores on (or through) the surface of a substrate can be formed using laser ablation, ion-beam milling, die punching, extrusion, solvent casting, or injection molding. Interior micropores can be provided by foaming the biomaterial using lyophilisation, supercritical solvent foaming, gas injection extrusion, casting with an extractable material (e.g., salts, sugar), layering of polymers onto a surface with micropores, or by other means known to those skilled in the art.

The biomaterial including a designed pattern of micropores may be provided in a sheet form. It is contemplated that a surgeon or other medical professional may readily trim or otherwise cut the biomaterial from a sheet of bulk material to match the configuration of a widened foramen, canal, or dissection region. Depending upon the substrate material selected, it is contemplated that the biomaterial can be further bent or shaped to match the particular configuration of the placement region. The device may also be rolled in a cuff shape or cylindrical shape to be used as, for example, a stent. Alternatively, the device could be pre-shaped or otherwise preformed into one or more patterns for subsequent use. However, a rectangular, square, round, or oblong shape formed of the biomaterial is a preferred embodiment of the invention. Typical lengths range from about 0.5 to about 5.0 centimeters (cm). Typical widths range from about 2 millimeters (mm) to about 1 cm. The overall thickness of the biomaterial generally ranges from about 0.25 mm to about 5 mm. It will be appreciated that greater or lesser thicknesses may be employed depending upon the application.

The biomaterial described herein can be used for a variety of different applications. Essentially, the biomaterial can be used for any implanted medical device. As such, the applications listed are exemplary, and should not be considered limiting. Applications of devices formed from the biomaterial include urinary suspensions, artificial esophagus or liner, a heart patch, lining for the pericardium, soft tissue onlay for plastic surgery, sheeting to cover large surface areas, shaped into nasal tips, artificial ears, subdermal tissue enhancement, injection sites for drugs or insulin, soft tissue filler, artificial tendon, plantar facia repair, reconstruction of cartilage, perivascular enhancements, patching or repairing a portion of a vessel, and tissue repair of bone, spine disc, articular cartilage, meniscus, fibrocartilage, tendons, ligaments, dura, skin, vascular grafts, nerves, liver, and pancreas. etc.

One application for the biomaterial is as a construction material for an implantable medical device. Alternately, or in addition, the biomaterial may be used to provide a covering (e.g., a coating) for an implantable medical device to improve the interaction with adjacent tissue. Surfaces that may be coated may include, for example, metals and alloys, ceramics, glasses, and polymers.

Examples of medical devices and medical materials that may be formed of or coated with the biomaterial include, but are not limited to, catheters, fibers, non-woven fabrics, vascular grafts, porous metals such as those used to reconstruct the acetabulum in revision, dental filling materials, materials for approximation, adhesion of tissues, materials used in osteo-synthesis (e.g. pins or bone screws), cardiac patches, sutures, soft and hard tissue scaffolds and fillers (e. g. collagen, calcium phosphate, bioglass), stents, bone void fillers intended for the repair of bone defects, intrauterine devices, root canal fillers, drug delivery pumps, implantable infusion pumps, spacer devices, implants containing medicinal products, vascular grafts, and heart valves.

The biomaterial may also be used to provide a tissue scaffold. Tissue scaffolding is a construct used as a support structure that allows tissue and/or cells to adhere, proliferate, and differentiate to form a healthy bone or tissue with restored functionality. Tissue scaffolds may either retain their shape and strength throughout the process of tissue repair/regeneration, or they may gradually disintegrate during tissue repair/regeneration. Use as tissue scaffolding is a preferred use of the biomaterial of the invention because the micropores provide a number of beneficial effects including, for example, encouraging tissue ingrowth, decreasing adverse biological reactions such as fibrous capsule formation, and texturing the surface of the tissue scaffolding to encourage retention of the tissue scaffolding material at the desired location.

Tissue scaffolds that include the biomaterial of the invention are generally shaped to define an interior surface of the body. The interior surface typically has an irregular three-dimensional topography. The tissue scaffold may be produced in a variety of shapes and sizes for the particular indication. One may select a non-absorbable scaffold for tissue defects that require permanent treatment and long-term durability and strength. Alternatively, one may select an absorbable scaffold for tissue defects that require only temporary treatment when one wants to avoid the potential complications associated with a permanent implant.

Tissue scaffolds can be produced in a variety of three-dimensional forms to facilitate sizing. An example is a scaffold with a curvature to construct a substantially cylindrical shape. A three dimensional structure could be machined using a system incorporating a third axis for micromachining. Alternatively, a sheet of biomaterial could be thermoformed into a three-dimensional shape after machining. When sheets of biomaterial are used, a layered scaffold in which the various layers may be of the same or different materials can be readily prepared. For example, one sheet could include micropores optimized to encourage device retention in one direction, while an overlaid sheet could include micropores optimized to encourage tissue growth. Such optimization could be obtained through varying the size of the micropores and/or including various drugs, as described herein.

The tissue scaffold can also include attachment regions, which may be adapted to receive sutures, staples or the like. Alternately, or in addition, tissue adhesive may be placed in the attachment regions. In addition, the individual layers for the tissue scaffold may have alignment regions to ensure the pores in the films match up properly.

Devices made of the biomaterial of the invention may be placed at the desired location within a surgical site by direct surgical placement or by endoscopic techniques. These devices provide, for example, a method of tissue reconstruction that includes surgically exposing a location in an individual's body where tissue is to be reconstructed and placing a device formed of the biomaterial of the invention at the location. A preferred technique for placing biomaterial within a surgical site when the biomaterial is compressible, such as biomaterial including a foamed polymer, is to introduce the biomaterial in a compressed state. The compressed biomaterial is inserted or otherwise placed at the desired location and then allowed to expand and thereby occupy the void or open region of the site. In order to maintain the biomaterial in a compressed state while it is being inserted, the biomaterial may be encapsulated in a water-soluble gelatin. Once the biomaterial has been placed at the desired location, the gelatin dissolves and allows the biomaterial to expand. Alternately, for non-compressible biomaterials, the biomaterial may be wrapped or bound in a water-soluble suture thread or film or other web or thread made of a material which is used to help fix the biomaterial and may safely be dissolved at the location.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

A series of experiments were conducted over a four-week time period in the dog animal model. The dog had implanted silicone biomaterial sheet with 500 micron pores with a 0.020 inch thickness that was laid onto the body dorsum of the dog's nose. The histological results indicated that there was complete tissue in-growth into the pores and incorporation of soft tissue as well as excellent vascularization with only a limited inflammatory response. Vasculature in some cases permeated the entire thickness of the membrane through the micropores extending from one surface to the other. In contrast, a control material without pores had classic fiber capsule formation around it with perivasculature outside the capsule and no integration of the implant with the surrounding tissue. There was no evidence of seroma formation, infection, or extrusion.

### Example 2

A series of experiments were also conducted over a four-week time period in the rabbit animal model. In the case of the rabbit animal model, a segment of ear cartilage was removed and replaced with two sheets of Silastic® biomaterial that had 250 micron pores through the sheeting. This was implanted subcutaneously as a cartilaginous replacement. In the rabbit experiment, the histologic results indicated no seroma formation had occurred post operatively with intimate integration of the soft tissue into and through the pores along with excellent vascularity throughout all the pores of the implant. Where some areas of the implant overlapped the rabbit's ear cartilage, the material displayed good tissue integration with no evidence of cartilaginous necroses or disruption of the cartilage microstructure. There was no evidence of seroma formation, infection, or extrusion.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A biomaterial including a substrate comprising designed pattern of micropores on one or more surfaces of the substrate.

2. The biomaterial of claim 1, wherein the substrate comprises a biocompatible polymer.

3. The biomaterial of claim 2, wherein the biocompatible polymer is a biodegradable polymer.

4. The biomaterial of claim 3, wherein the biodegradable polymer is selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups poly(anhydrides), polyphosphazenes, tyrosine derived polycarbonates, and biopolymers.

5. The biomaterial of claim 1, wherein the micropores comprise continuous micropores.

6. The biomaterial of claim 1, wherein the diameter of the micropores is from about 10 microns to about 5,000 microns.

7. The biomaterial of claim 4, wherein the diameter of the micropores is from about 100 to about 1,000 microns.

8. The biomaterial of claim 1, wherein the micropores retain a pharmacologically active agent.

9. The biomaterial of claim 8, wherein the pharmacologically active agent is retained in the micropores by a retaining material.

10. The biomaterial of claim 8, wherein a laminate layer is provided on one or more surface of the substrate over the micropores.

11. The biomaterial of claim 1, wherein the designed pattern comprises an uneven pattern of spaced micropores that allow for asymmetrical expansion of the biomaterial.

12. The biomaterial of claim 1, wherein the surface of the substrate further comprises texturing.

13. The biomaterial of claim 1, wherein the designed pattern of micropores is a regular pattern.

14. The biomaterial of claim 1, wherein the designed pattern of micropores is a gradient pattern.

15. The biomaterial of claim 1, wherein the biomaterial comprises a sheet.

16. The biomaterial of claim 1, wherein the biomaterial comprises a plurality of layers of biocompatible polymers.

17. The biomaterial of claim 1, wherein the biomaterial comprises a tissue scaffold.

18. A method of tissue reconstruction, comprising surgically exposing a location in an individual's body where tissue is to be reconstructed and placing the tissue scaffolding material of claim 17 at the location.

19. The method of claim 18, wherein the tissue to be reconstructed is cartilage.

20. The method of claim 19, wherein the tissue comprises a nose or an ear.
